(19) United States
(12) Patent Application Publication
(10) Pub. No.: US 2018/0154127 A1
(43) Pub. Date: Jun. 7, 2018

(54) COMBINATIONAL EYEBROW KIT

(71) Applicant: Khoi Dang, Boilingbrook, IL (US)

(72) Inventor: Khoi Dang, Boilingbrook, IL (US)

(21) Appl. No.: 15/369,608

(22) Filed: Dec. 5, 2016

(51) Int. Cl. A61M 37/00; A45D 40/00

(52) U.S. Cl. CPC A61M 37/0084; A45D 40/00

(57) ABSTRACT

A method of using a combinational eyebrow kit to make a sharpened eyebrow structure for a person is disclosed. The method comprises creating a hair stroke by selectively implanting a pigment in a line under the skin by a three-dimensional blading device to mimic a plurality of hair structures and shading the hair structure using a tattoo applicator in a predetermined intensity to make a sharpened eyebrow structure. The combinational eyebrow kit is configured to provide a semi-permanent technique that uses the three-dimensional blading device to implant the pigments to fill out the hairless gaps in the eyebrow structure.

### BACKGROUND

Women work to enhance their beauty and appearance on a regular basis. The appearance of their eyebrows plays a significant role in highlighting their attractiveness and use both simulations and natural augmentation to get the desired look for their eyebrow structure. Some women opt to go for tattooing to simplify their make-up procedure. However, it takes manual efforts for a user to make a full and complete look for their individual eyebrow structure. Most of the tattooing procedures give only a temporary result and with time, the appearance of the eyebrow structure starts fading

Previous attempts have been made to provide a blading procedure by moving the device across the eyebrow to form a needle implantation pattern. Even though the results are either permanent or semi-permanent, there are more possibilities for the person to pick, peel and scratch the finished eyebrow as the healing process takes time. Alternate procedures such as pencils, powers, gels, and tattooing are available on the market to produce desired results for their eyebrow structure. However, not all the devices and methods produce efficient and desired results. Sometimes, these devices can also cause injury to the eyebrow skin and infections. Recovery time for such injuries normally takes time to heal and therefore not all of those methods are practical.

Prior art reference EP 2011539 B1 discloses a needle for tattooing skin or eyebrow structure to improve the efficiency of drawing of lines or filling the color area of a tattoo pattern. However, it does not customize the eyebrow structure as desired. The invention provides only temporary make up for the eyebrow structure.

In light of the aforementioned drawbacks and limitations, there exists a need for a combinational eyebrow kit to make a sharpened eyebrow structure both customized and natural in appearance.

### SUMMARY

The objective of the present invention is to provide a combinational eyebrow kit for a person and a method of using the combinational eyebrow kit to make a sharpened eyebrow structure.

According to one embodiment of the present invention, the method comprises the steps of creating a hair stroke by selectively implanting a pigment in a line under the skin by a three-dimensional blading device to mimic a plurality of hair structures and shading the hair structure using a tattoo applicator in a predetermined intensity to make a sharpened eyebrow structure.

According to another embodiment of the present invention, the combinational eyebrow kit provides a semi-permanent technique to create the sharpened eyebrow structure using three-dimensional blading device to implant the pigments to fill out the hairless gaps in the eyebrow structure.

### BRIEF DESCRIPTIONS OF SEVERAL VIEWS OF DRAWINGS

FIG. 1 illustrates a sharpened eyebrow structure made using a combinational eyebrow kit for a person, incorporating the aspects of the present invention.

FIG. 2A illustrates a three-dimensional blading device of the combinational eyebrow kit, incorporating the aspects of the present invention.

FIG. 2B illustrates a tattoo applicator of the combinational eyebrow kit, incorporating the aspects of the present invention.

FIG. 3 is a flowchart illustrating the method of using the combinational eyebrow kit, incorporating the aspects of the present invention.

### DETAILED DESCRIPTION OF DIFFERENT EMBODIMENTS

The present invention is related to a combinational eyebrow kit 100 for a person and a method of using the combinational eyebrow kit 200 to make a sharpened eyebrow structure 108.

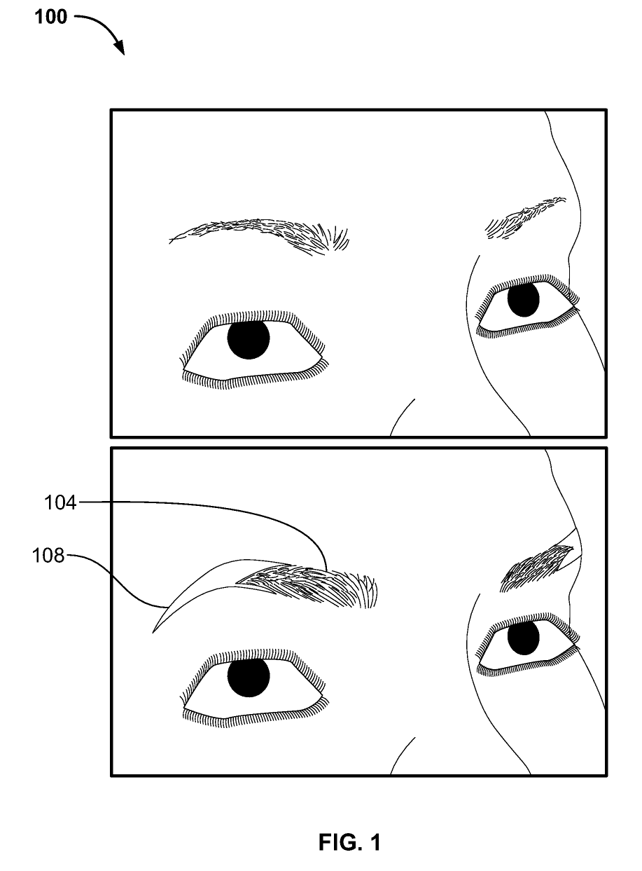

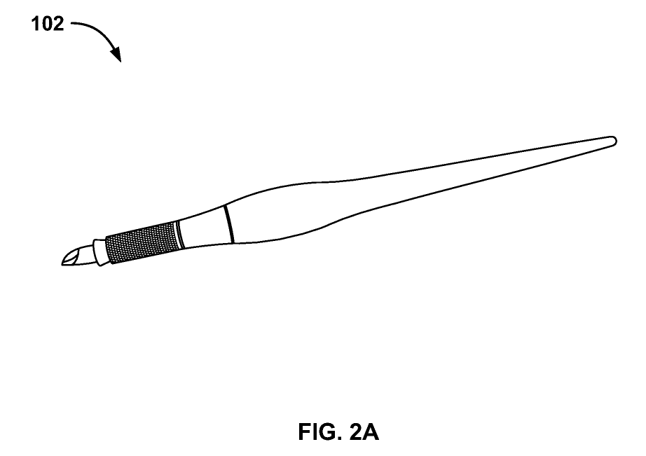

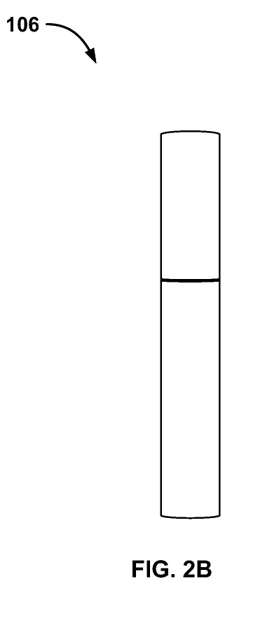

According to an embodiment as shown in FIG. 1, which illustrates a sharpened eyebrow structure 108 made using a combinational eyebrow kit 100 for a person. The combinational eyebrow kit 100 is configured to provide a three-dimensional blading device 102 to mimic a plurality of hair structures 104 by creating a hair stroke by selectively implanting a pigment in a line under the skin as shown in FIG. 2. Then, a tattoo applicator 106 is used to shade the hair structure 104 in a predetermined intensity to make a sharpened eyebrow structure 108. The three-dimensional blading device 102 and the tattoo applicator 106 are made from a lightweight and durable plastic material.

In an embodiment as shown in FIG. 2A, the combinational eyebrow kit 100 uses a semi-permanent technique to create the sharpened eyebrow structure 108 for a person. The three-dimensional blading device 102 comprises a pen like structure having a micro blade at one end configured to draw individual strokes one by one and selectively implanting a pigment in a line under the skin. The three-dimensional blading device 102 is primarily used to do at least one of a reconstruct, define, cover gaps or fill-in over plucked brows. In preferred embodiments, the three-dimensional blading device 102 is a micro-blading device configured to implant the pigments to fill out the hairless gaps in the eyebrow structure 108.

In an embodiment as shown in FIG. 2B, the tattoo applicator 106 is configured to shade a gradient finish to make the sharpened eyebrow structure 108. The tattoo applicator 106 could be selected from any of the available devices in the market such as a self-powered device or a manual power supply means. However, the tattoo applicator 106 is of compact size for easier and long-time holding by a user during the application. If the device 100 is self-powered, it is configured to have a rechargeable battery and the tattoo applicator 106 comprises a removable cap to expose the tattooing needle during the use. Depending upon the requirements, the color of the tattooing could be varied by changing the dye liquid to be applied on the eyebrow structure 108. In some embodiments, the tattoo applicator 106 could be used to make a gradient finish to make the sharpened eyebrow structure 108. The gradient finish further enhances the appearance of the eyebrow structure 108 as the geometry of the eyebrow structure 108 is made to suit the person's eye shape. This combined procedure for the process provides natural feathering of hair like strokes directly on the skin's surface that creates natural looking eyebrow structure 108.

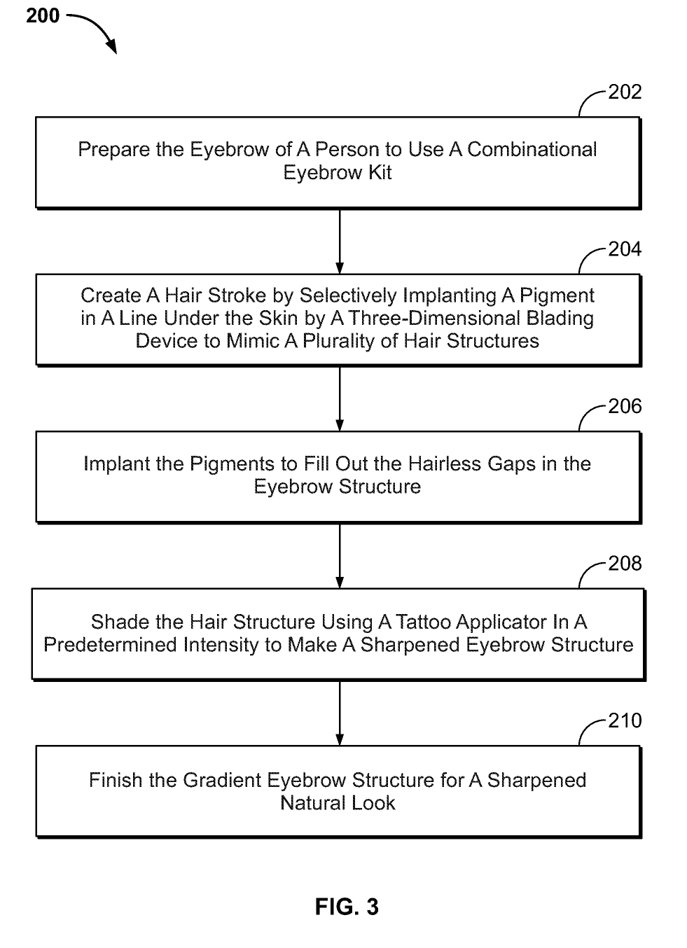

In a preferred embodiment as shown in FIG. 3, the method of using a combinational eyebrow kit 200 for the person to make the sharpened eyebrow structure 108 is disclosed. The method comprises the following: at step 202, preparing the eyebrows of the person to use the combinational eyebrow kit 100 and planning the customized eyebrow structure 108 for them based on the requirements. Before starting the procedure, the skin must be kept as numb as much as possible to avoid discomfort. The user takes all the necessary measurements related to the person's face shape and structure. For example, the thickness of the brow and the hairless gaps in the eyebrow structure 108 are predetermined. At step 204, creating a hair stroke by selectively implanting a pigment in a line under the skin by a three-dimensional blading device 102 to mimic a plurality of hair structures 104. This procedure is carried out with predetermined parameters to adapt the different skin types for better results. The hair stroke is done precisely to mimic the exact structure of the hair. The pigment is selectively implanted in a plurality of colors based on the user requirements for the eyebrow structure 108. At step 206, the hairless gaps in the eyebrow structure 108 is identified and the implanting is gently carried out with care. Once this procedure is done, a post-care medication could be done to smoothen the eyebrow structure 108. After a predefined time, at step 208, the shading of the hair structure 104 using the tattoo applicator 106 in a predetermined intensity to make the sharpened eyebrow structure 108. The tattoo applicator 106 applies the tattoo in a predetermined intensity ranging from light to dark shades to make the sharpened eyebrow structure 108. At step 210, the gradient finish for the eyebrow structure 108 is carried out based on the geometric parameters to give a sharpened and stylish look for the eyebrow structure 108 of the person.

The combinational eyebrow kit, according to the present invention, improves the full and complete look of the eyebrow structure 108 of the person. The combinational eyebrow kit 100 uses both the three-dimensional blading device 102 and the tattoo applicator 106, one after another to give an enhanced look and appearance for the eyebrow structure 108 of the person. The results of the eyebrow structure 108 using this combinational eyebrow kit 100 could last for more than 18. The three-dimensional blading device 102 does not provide as much discomfort to the person during the process and the healing process is shorter. The embroidery strokes and tattooing provides an unparalleled natural finish with the appearance of real hair structure 104.

Although the present invention has been described herein in the context of a particular implementation in a particular environment for a particular purpose, those of ordinary skill in the art will recognize that its usefulness is not limited thereto and that the present invention may be beneficially implemented in any number of environments for any number of purposes. Accordingly, the claims set forth below should be construed in view of the full breadth and spirit of the present invention as described herein.

I claim:

1. A method of using a combinational eyebrow kit for a person, the method comprising:
creating, a hair stroke by selectively implanting a pigment in a line under the skin by a three-dimensional blading device to mimic a plurality of hair structures and shading, the hair structure using a tattoo applicator in a predetermined intensity to make a sharpened eyebrow structure.

2. The method of claim 1, is a semi-permanent technique to create the sharpened eyebrow structure.

3. The method of claim 1, wherein the three-dimensional blading device is configured to implant the pigments to fill out the hairless gaps in the eyebrow structure.

4. The method of claim 1, wherein the pigment is selectively implanted in a plurality of colors based on the user requirements for the eyebrow structure.

5. The method of claim 1, wherein the tattoo applicator is configured to shade a gradient finish to make the sharpened eyebrow structure.

6. The method of claim 1, wherein the tattoo applicator applies the tattoo in a predetermined intensity ranging from light to dark shades to make the sharpened eyebrow structure.

7. The method of claim 1, wherein the predetermined geometric attributes of the person's eye are measured during the three-dimensional blading process for customized results.

8. The method of claim 1, wherein the three-dimensional blading device and the tattoo applicator are made from a lightweight and durable plastic material.
